(19) United States
(12) Patent Application Publication
(10) Pub. No.: US 2003/0022311 A1
(43) Pub. Date: Jan. 30, 2003

(54) Human CIS protein

(76) Inventors: Damien D. Dunnington, Lafayette Hill, PA (US); James D. Frantz, Newton, MA (US); Steven E. Shoelson, Natick, MA (US)

Correspondence Address: GLAXOSMITHKLINE, Corporate Intellectual Property - UW2220, P.O. Box 1539, King of Prussia, PA, 19406-0939, US

(21) Appl. No.: 10/262,130

(22) Filed: Oct. 1, 2002

Related U.S. Application Data
(63) Continuation of application No. 08/655,327, filed on May 21, 1996, now abandoned.

(51) Int. Cl. C12P 21/02; C12N 5/06; C07K 14/435; C07H 21/04

(52) U.S. Cl. 435/69.1; 435/320.1; 435/325; 530/350; 536/23.5

(57) ABSTRACT

Isolated nucleic acid encoding a human cytokine-inducible SH2-containing protein, protein obtainable from the nucleic acid, recombinant host cells transformed with the nucleic acid and use of the protein and nucleic acid sequence are disclosed.

### FIELD OF THE INVENTION

The present invention relates to an isolated human cytokine-inducible SH2-containing (CIS) gene; to essentially pure human CIS protein; and to compositions and methods of producing and using human CIS sequences and proteins.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein tyrosine kinases (PTK) and phosphatases. Tyrosine phosphorylation is a major mediator of signal transduction in multicellular organisms. Receptor-bound, membrane-bound and intracellular PTKs regulate cell proliferation, cell differentiation and signalling processes in hematopoietic cells.

Aberrant protein tyrosine kinase activity has been implicated or is suspected in a number of pathologies such as diabetes, atherosclerosis, psoriasis, septic shock, bone loss, anemia, many cancers and other proliferative diseases. Accordingly, tyrosine kinases and the signal transduction pathways which they are part of are potential targets for drug design. For a review, see Levitzki et al. in Science 267, 1782-1788 (1995).

Many of the proteins comprising signal transduction pathways are present at low levels and often have opposing activities. The properties of these signalling molecules allow the cell to control transduction by means of the subcellular location and juxtaposition of effectors as well as by balancing activation with repression such that a small change in one pathway can achieve a switching effect.

The formation of transducing complexes by juxtaposition of the signalling molecules through protein-protein interactions are mediated by specific docking domain sequence motifs. Src homology 2 (SH2) domains, which are conserved non-catalytic sequences of approximately 100 amino acids found in a variety of signalling molecules such as non-receptor PTKs and kinase target effector molecules and in oncogenic proteins, play a critical role. The SH2 domains are highly specific for short phosphotyrosine-containing peptide sequences found in autophosphorylated PTK receptors or intracellular tyrosine kinases.

One approach towards the pharmacological regulation of signal transduction pathways is to design inhibitory ligands which selectively bind to a chosen SH2 domain and thus block the interaction of a phosphorylated protein tyrosine kinase with its SH2-containing target molecule, thereby disrupting signal transduction. Any selective inhibitors would provide a useful lead for drug development.

Cytokine-inducible SH2-containing protein, otherwise known as CIS or SIC, is an SH2 domain containing protein identified in the mouse as an early response gene induced by certain cytokines such as interleukins 2 and 3, granulocyte-macrophage colony stimulating factor and erythropoietin (EPO). See Yoshimura et al., EMBO J. 14, 2816-2826 (1995). CIS is expressed in liver, kidney, heart, stomach and lung tissues. It binds to the tyrosine-phosphorylated IL3 or EPO receptors and when overexpressed, inhibits signal transduction through these receptors. CIS appears to belong to the “adaptor” class of SH2-containing proteins and may function by recruitment of negative regulators of signaling such as phosphatases or by masking binding sites for positive effectors. Inactivation of CIS may be expected to enhance signaling through the IL-3 or EPO receptors, thereby up-regulating the effects of these cytokines. In the case of EPO, such up-regulation may have utility as a means of stimulating hematopoiesis. Therefore, specific inhibitors of CIS may be useful in the treatment of anemia.

Binding of CIS to cytokine receptors is mediated by SH2-phosphotyrosine interactions, which are amenable to disruption by small molecule agents. Discovery of such agents is best carried out using the human CIS molecule or fragment thereof, and an appropriate ligand such as the tyrosine-phosphorylated EPO receptor or a synthetic phosphopeptide. Thus, a need exists for provision of the nucleotide and amino acid sequences corresponding to human CIS, for compounds which modulate the activity of CIS homologs and isoforms, for methods to identify such modulators and for reagents useful in such methods.

### SUMMARY OF THE INVENTION

Accordingly, one aspect of the present invention is an isolated polynucleotide selected from the group consisting of:

(a) a polynucleotide encoding human CIS having the nucleotide sequence as set forth in SEQ ID NO:1 from nucleotide 72 to 846;

(b) a polynucleotide capable of hybridizing to the complement of a polynucleotide according to (a) under moderately stringent hybridization conditions and which encodes a functional human CIS; and

(c) a degenerate polynucleotide according to (a) or (b).

Another aspect of the invention is a functional polypeptide encoded by the polynucleotides of the invention.

Another aspect of the invention is a method for preparing essentially pure human CIS protein comprising culturing a recombinant host cell comprising a vector comprising a polynucleotide of the invention under conditions promoting expression of the protein and recovery thereof.

Another aspect of the invention is an antisense oligonucleotide comprising a sequence which is capable of binding to the polynucleotide of the invention.

Another aspect of the invention is a modulator of the polypeptides of the invention.

Another aspect of the invention is a method for assaying a medium for the presence of a substance that modulates CIS activity comprising the steps of:

(a) providing a CIS protein having the amino acid sequence of CIS (SEQ ID NO:2) or a functional derivative thereof and a cellular binding partner or synthetic analog thereof;

(b) incubating with a test substance which is suspected of modulating CIS activity under conditions which permit the formation of a CIS protein/cellular binding partner complex;

(c) assaying for the presence of the complex, free CIS protein or free cellular binding partner; and

(d) comparing to a control to determine the effect of the substance.

Another aspect of the invention is a method for assaying for the presence of a substance that modulates CIS activity by direct binding to CIS protein comprising the steps of:

(a) providing a labelled CIS protein having the amino acid sequence of CIS (SEQ ID NO:2) or a functional derivative thereof;

(b) providing solid support-associated modulator candidates;

(c) incubating a mixture of the labelled CIS protein with the support-associated modulator candidates under conditions which can permit the formation of a CIS protein/modulator candidate complex;

(d) separating the solid support from free soluble labelled CIS protein;

(e) assaying for the presence of solid support-associated labelled protein;

(f) isolating the solid support complexed with labelled CIS protein; and

(g) identifying the modulator candidate.

Another aspect of the invention is CIS protein modulating compounds identified by the methods of the invention.

Another aspect of the invention is a method for the treatment of a patient having need to modulate CIS activity comprising administering to the patient a therapeutically effective amount of the modulating compounds of the invention.

### BRIEF DESCRIPTION OF THE DRAWING

FIG. 1 is an amino acid sequence alignment of human CIS with murine CIS.

### DETAILED DESCRIPTION OF THE INVENTION

As used herein, the term “CIS gene” refers to DNA molecules comprising a nucleotide sequence that encodes human CIS. The CIS gene sequence is listed in SEQ ID NO:1. The coding region of the CIS gene consists of nucleotides 72-846 of SEQ ID NO:1. The deduced 258 amino acid sequence of the gene product CIS is listed in SEQ ID NO:2.

As used herein, the term “functional fragments” when used to modify a specific gene or gene product means a less than full length portion of the gene or gene product which retains substantially all of the biological function associated with the full length gene or gene product to which it relates. An example of a functional fragment of human CIS is the isolated SH2 domain lacking flanking sequences. To determine whether a fragment of a particular gene or gene product is a functional fragment, fragments are generated by well-known nucleolytic or proteolytic techniques or by the polymerase chain reaction and the fragments tested for the described biological function.

As used herein, an “antigen” refers to a molecule containing one or more epitopes that will stimulate a host's immune system to make a humoral and/or cellular antigen-specific response. The term is also used herein interchangeably with “immunogen.”

As used herein, the term “epitope” refers to the site on an antigen or hapten to which a specific antibody molecule binds. The term is also used herein interchangeably with “antigenic determinant” or “antigenic determinant site.”

As used herein, “monoclonal antibody” is understood to include antibodies derived from one species (e.g., murine, rabbit, goat, rat, human, etc.) as well as antibodies derived from two (or perhaps more) species (e.g., chimeric and humanized antibodies).

As used herein, a coding sequence is “operably linked to” another coding sequence when RNA polymerase will transcribe the two coding sequences into a single mRNA, which is then translated into a single polypeptide having amino acids derived from both coding sequences. The coding sequences need not be contiguous to one another so long as the expressed sequence is ultimately processed to produce the desired protein.

As used herein, “recombinant” polypeptides refer to polypeptides produced by recombinant DNA techniques; i.e., produced from cells transformed by an exogenous DNA construct encoding the desired polypeptide. “Synthetic” polypeptides are those prepared by chemical synthesis.

As used herein, a “replicon” is any genetic element (e.g., plasmid, chromosome, virus) that functions as an autonomous unit of DNA replication in vivo; i.e., capable of replication under its own control.

As used herein, a “vector” is a replicon, such as a plasmid, phage, or cosmid, to which another DNA segment may be attached so as to bring about the replication of the attached segment.

As used herein, a “reference” gene refers to the wild type human CIS gene sequence of the invention and is understood to include the various sequence polymorphisms that exist, wherein nucleotide substitutions in the gene sequence exist, but do not affect the essential function of the gene product.

As used herein, a “mutant” gene refers human CIS sequences different from the reference gene wherein nucleotide substitutions and/or deletions and/or insertions result in perturbation of the essential function of the gene product.

As used herein, a DNA “coding sequence of” or a “nucleotide sequence encoding” a particular protein, is a DNA sequence which is transcribed and translated into a polypeptide when placed under the control of appropriate regulatory sequences.

As used herein, a “promoter sequence” is a DNA regulatory region capable of binding RNA polymerase in a cell and initiating transcription of a downstream (3′ direction) coding sequence. For purposes of defining the present invention, the promoter sequence is bound at its 3′ terminus by a translation start codon (e.g., ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

As used herein, DNA “control sequences” refers collectively to promoter sequences, ribosome binding sites, polyadenylation signals, transcription termination sequences, upstream regulatory domains, enhancers and the like, which collectively provide for the expression (i.e., the transcription and translation) of a coding sequence in a host cell.

As used herein, a control sequence “directs the expression” of a coding sequence in a cell when RNA polymerase will bind the promoter sequence and transcribe the coding sequence into mRNA, which is then translated into the polypeptide encoded by the coding sequence.

As used herein, a “host cell” is a cell which has been transformed or transfected, or is capable of transformation or transfection by an exogenous DNA sequence.

As used herein, a cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA.

As used herein, “transfection” or “transfected” refers to a process by which cells take up foreign DNA and integrate that foreign DNA into their chromosome. Transfection can be accomplished, for example, by various techniques in which cells take up DNA (e.g., calcium phosphate precipitation, electroporation, assimilation of liposomes, etc.) or by infection, in which viruses are used to transfer DNA into cells.

As used herein, a “target cell” is a cell that is selectively transfected over other cell types (or cell lines).

As used herein, a “clone” is a population of cells derived from a single cell or common ancestor by mitosis. A “cell line” is a clone of a primary cell that is capable of stable growth in vitro for many generations.

As used herein, a “heterologous” region of a DNA construct is an identifiable segment of DNA within or attached to another DNA molecule that is not found in association with the other molecule in nature. Thus, when the heterologous region encodes a gene, the gene will usually be flanked by DNA that does not flank the gene in the genome of the source animal. Another example of a heterologous coding sequence is a construct where the coding sequence itself is not found in nature (e.g., synthetic sequences having codons different from the native gene). Allelic variation or naturally occurring mutational events do not give rise to a heterologous region of DNA, as used herein.

As used herein, a “modulator” of a polypeptide is a substance which can affect the polypeptide function.

An aspect of the present invention is isolated polynucleotides encoding a human CIS protein and substantially similar sequences. Isolated polynucleotide sequences are substantially similar if they are capable of hybridizing under moderately stringent conditions to SEQ ID NO:1 or they encode DNA sequences which are degenerate to SEQ ID NO:1 or are degenerate to those sequences capable of hybridizing under moderately stringent conditions to SEQ ID NO:1.

Moderately stringent conditions is a term understood by the skilled artisan and has been described in, for example, Sambrook et al. Molecular Cloning: A Laboratory Manual, 2nd edition, Vol. 1, pp. 101-104, Cold Spring Harbor Laboratory Press (1989). An exemplary hybridization protocol using moderately stringent conditions is as follows. Nitrocellulose filters are prehybridized at 65° C. in a solution containing 6X SSPE, 5X Denhardt's solution (10g Ficoll, 10g BSA and 10g polyvinylpyrrolidone per liter solution), 0.05% SDS and 100 ug/ml tRNA. Hybridization probes are labeled, preferably radiolabelled (e.g., using the Bios TAG-IT® kit). Hybridization is then carried out for approximately 18 hours at 65° C. The filters are then washed twice in a solution of 2X SSC and 0.5% SDS at room temperature for 15 minutes. Subsequently, the filters are washed at 58° C., air-dried and exposed to X-ray film overnight at −70° C. with an intensifying screen.

Degenerate DNA sequences encode the same amino acid sequence as SEQ ID NO:2 or the proteins encoded by that sequence capable of hybridizing under moderately stringent conditions to SEQ ID NO:1, but have variation(s) in the nucleotide coding sequences because of the degeneracy of the genetic code. For example, the degenerate codons UUC and UUU both code for the amino acid phenylalanine, whereas the four codons GGX all code for glycine.

Alternatively, substantially similar sequences are defined as those sequences in which about 66%, preferably about 75% and most preferably about 90%, of the nucleotides or amino acids match over a defined length of the molecule. As used herein, substantially similar refers to the sequences having similar identity to the sequences of the instant invention. Thus nucleotide sequences that are substantially the same can be identified by hybridization or by sequence comparison. Protein sequences that are substantially the same can be identified by techniques such as proteolytic digestion, gel electrophoresis and/or microsequencing. Excluded from the definition of substantially similar sequences is the murine CIS gene.

Embodiments of the isolated polynucleotides of the invention include DNA, genomic DNA and RNA, preferably of human origin. A method for isolating a nucleic acid molecule encoding a CIS protein is to probe a genomic or cDNA library with a natural or artificially designed probe using art recognized procedures. See, e.g., “Current Protocols in Molecular Biology”, Ausubel et al. (eds.) Greene Publishing Association and John Wiley Interscience, New York, 1989,1992. The ordinarily skilled artisan will appreciate that SEQ ID NO:1 or fragments thereof comprising at least 15 contiguous nucleotides are particularly useful probes. It is also appreciated that such probes can be and are preferably labeled with an analytically detectable reagent to facilitate identification of the probe. Useful reagents include, but are not limited to, radioisotopes, fluorescent dyes or enzymes capable of catalyzing the formation of a detectable product. The probes would enable the ordinarily skilled artisan are to isolate complementary copies of genomic DNA, cDNA or RNA polynucleotides encoding CIS proteins from human, mammalian or other animal sources or to screen such sources for related sequences, e.g., additional members of the family, type and/or subtype, including transcriptional regulatory and control elements as well as other stability, processing, translation and tissue specificity-determining regions from 5′ and/or 3′ regions relative to the coding sequences disclosed herein, all without undue experimentation.

Another aspect of the invention is functional polypeptides encoded by the polynucleotides of the invention. An embodiment of a functional polypeptide of the invention is the human CIS protein having the amino acid sequence set forth in SEQ ID NO:2.

Another aspect of the invention is a method for preparing essentially pure human CIS protein. Yet another aspect is the human CIS protein produced by the preparation method of the invention. This protein has the amino acid sequence listed in SEQ ID NO:2 and includes variants with a substantially similar amino acid sequence that have the same function. The proteins of this invention are preferably made by recombinant genetic engineering techniques by culturing a recombinant host cell containing a vector encoding the polynucleotides of the invention under conditions promoting the expression of the protein and recovery thereof.

The isolated polynucleotides, particularly the DNAs, can be introduced into expression vectors by operatively linking the DNA to the necessary expression control regions, e.g., regulatory regions, required for gene expression. The vectors can be introduced into an appropriate host cell such as a prokaryotic, e.g., bacterial, or eukaryotic, e.g., yeast or mammalian cell by methods well known in the art. See Ausubel et al., supra. The coding sequences for the desired proteins, having been prepared or isolated, can be cloned into any suitable vector or replicon. Numerous cloning vectors are known to those of skill in the art and the selection of an appropriate cloning vector is a matter of choice. Examples of recombinant DNA vectors for cloning and host cells which they can transform include, but are not limited to, the bacteriophage (E. coli), pBR322 (E. coli), pACYC177 (E. coi), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), a baculovirus insect cell system, a Drosophila insect system, YCp19 (Saccharomyces) and pSV2neo (mammalian cells). See generally, “DNA Cloning”: Vols. I & II, Glover et al. ed. Press Oxford (1985) (1987); and T. Maniatis et al. (“Molecular Cloning” Cold Spring Harbor Laboratory (1982).

The gene can be placed under the control of control elements such as a promoter, ribosome binding site (for bacterial expression) and, optionally, an operator, so that the DNA sequence encoding the desired protein is transcribed into RNA in the host cell transformed by a vector containing the expression construct. The coding sequence may or may not contain a signal peptide or leader sequence. The proteins of the present invention can be expressed using, for example, the E. coli tac promoter or the protein A gene (spa) promoter and signal sequence. Leader sequences can be removed by the bacterial host in post-translational processing. See, e.g., U.S. Pat. Nos. 4,431,739; 4,425,437 and 4,338,397.

In addition to control sequences, it may be desirable to add regulatory sequences which allow for regulation of the expression of the protein sequences relative to the growth of the host cell. Regulatory sequences are known to those of skill in the art. Exemplary are those which cause the expression of a gene to be turned on or off in response to a chemical or physical stimulus, including the presence of a regulatory compound or to various temperature or metabolic conditions. Other types of regulatory elements may also be present in the vector, for example, enhancer sequences.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences, i.e., RNA polymerase which binds to the DNA molecule at the control sequences transcribes the coding sequence. Modification of the sequences encoding the particular antigen of interest may be desirable to achieve this end. For example, in some cases it may be necessary to modify the sequence so that it may be attached to the control sequences with the appropriate orientation; i.e., to maintain the reading frame. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases, it may be desirable to produce mutants or analogues of human CIS protein. Mutants or analogues may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., T. Maniatis et al., supra; “DNA Cloning,” Vols. I and II, supra; and “Nucleic Acid Hybridization”, supra.

Depending on the expression system and host selected, the proteins of the present invention are produced by growing host cells transformed by an expression vector described above under conditions whereby the protein of interest is expressed. Preferred mammalian cells include human embryonic kidney cells (293), monkey kidney cells, fibroblast (COS) cells, Chinese hamster ovary (CHO) cells, Drosophila or murine L-cells. If the expression system secretes the protein into growth media, the protein can be purified directly from the media. If the protein is not secreted, it is isolated from cell lysates or recovered from the cell membrane fraction. The selection of the appropriate growth conditions and recovery methods are within the skill of the art.

An alternative method to identify proteins of the present invention is by constructing gene libraries, using the resulting clones to transform E. coli and pooling and screening individual colonies using polyclonal serum or monoclonal antibodies to human CIS.

The proteins of the present invention may also be produced by chemical synthesis such as solid phase peptide synthesis on an automated peptide synthesizer, using known amino acid sequences or amino acid sequences derived from the DNA sequence of the genes of interest. Such methods are known to those skilled in the art.

The proteins of the present invention or their fragments comprising at least one epitope can be used to produce antibodies, both polyclonal and monoclonal, directed to epitopes corresponding to amino acid sequences disclosed herein. If polyclonal antibodies are desired, a selected mammal such as a mouse, rabbit, goat or horse is immunized with a protein of the present invention, or its fragment, or a mutant protein. Serum from the immunized animal is collected and treated according to known procedures. Serum polyclonal antibodies can be purified by immunoaffinity chromatography or other known procedures.

Monoclonal antibodies to the proteins of the present invention, and to the fragments thereof, can also be readily produced by one skilled in the art. The general methodology for making monoclonal antibodies by using hybridoma technology is well known. Immortal antibody-producing cell lines can be created by cell fusion and also by other techniques such as direct transformation of B lymphocytes with oncogenic DNA or transfection with Epstein-Barr virus. See, e.g., M. Schreier et al., “Hybridoma Techniques” (1980); Hammerling et al., “Monoclonal Antibodies and T-cell Hybridomas” (1981); Kennett et al., “Monoclonal Antibodies” (1980); and U.S. Pat. Nos. 4,341,761; 4,399,121; 4,427,783; 4,444,887; 4,452,570; 4,466,917; 4,472,500; 4,491,632; and 4,493,890. Panels of monoclonal antibodies produced against the antigen of interest, or fragment thereof, can be screened for various properties, i.e., for isotype, epitope, affinity, etc. Monoclonal antibodies are useful in purification, using immunoaffinity techniques, of the individual antigens which they are directed against. Alternatively, genes encoding the monoclonals of interest may be isolated from the hybridomas by PCR techniques known in the art and cloned and expressed in the appropriate vectors. The antibodies of this invention, whether polyclonal or monoclonal have additional utility in that they may be employed as reagents in immunoassays, RIA, ELISA, and the like. The antibodies of the invention can be labeled with an analytically detectable reagent such as a radioisotope, fluorescent molecule or enzyme.

Chimeric antibodies, in which non-human variable regions are joined or fused to human constant regions (see, e.g., Liu et al., Proc. Natl Acad. Sci. USA, 84, 3439 (1987)), may also be used in assays or therapeutically. Preferably, a therapeutic monoclonal antibody would be “humanized” as described in Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9:, 421 (1991).

Another aspect of the present invention is modulators of the polypeptides of the invention. Functional modulation of CIS by a substance includes partial to complete inhibition of function, identical function, as well as enhancement of function. Embodiments of modulators of the invention include peptides, oligonucleotides and small organic molecules including peptidomimetics.

Another aspect of the invention is antisense oligonucleotides comprising a sequence which is capable of binding to the polynucleotides of the invention. Synthetic oligonucleotides or related antisense chemical structural analogs can be designed to recognize, specifically bind to and prevent transcription of a target nucleic acid encoding CIS protein by those of ordinary skill in the art. See generally, Cohen, J. S., Trends in Pharm. Sci., 10, 435(1989) and Weintraub, H. M., Scientific American, January (1990) at page 40.

Another aspect of the invention is a method for assaying a medium for the presence of a substance that modulates CIS protein function by affecting the binding of CIS protein to cellular binding partners. Examples of modulators include, but are not limited to peptides and small organic molecules including peptidomimetics. A CIS protein is provided having the amino acid sequence of human CIS (SEQ ID NO:2) or a functional derivative thereof together with a cellular binding partner or synthetic analog thereof. The mixture is incubated with a test substance which is suspected of modulating CIS activity, under conditions which permit the formation of a CIS gene product/cellular binding partner complex. An assay is performed for the presence of the complex, free CIS protein or free cellular binding partner and the result compared to a control to determine the effect of the test substance.

Another aspect of the invention is a method for assaying for the presence of a substance that modulates CIS activity by direct binding to CIS protein. Examples of modulators include, but are not limited to, peptides and small organic molecules including peptidomimetics. Modulator candidates are synthesized on a solid support by techniques such as those disclosed in Lam et al., Nature 354, 82 (1991) or Burbaum et al., Proc. Natl. Acad. Sci. USA 92, 6027 (1995) to provide solid support-associated modulator candidates. A labelled CIS protein is provided having the amino acid sequence of human CIS (SEQ ID NO:2) or a functional derivative thereof. Exemplary labels include directly attached fluorescent or colored dyes, biotin, radioisotopes or epitope tags, which are detectable by a suitable antibody. A mixture of solid support-associated modulator candidates and labelled CIS protein is incubated under conditions which can permit the formation of a CIS protein/modulator candidate complex. The solid support is separated from free soluble labelled CIS protein. An assay is performed for the presence of solid support-associated labelled protein. Solid supports complexed with labelled protein are isolated and the identity of the modulator candidate determined by techniques well known to those skilled in the art, such as the TOF-SIMS method in Brummel et al., Science 264, 399-402(1994).

Modulation of CIS function would be expected to have effects on hematopoietic function. Any antagonist modulators so identified would be expected to have up-regulatory effects on the cytokines IL-3 or EPO and be useful as a therapeutic for the treatment and prevention of anemia.

Further, CIS could be used to isolate proteins which interact with it and this interaction could be a target for interference. Inhibitors of protein-protein interactions between CIS and other factors could lead to the development of pharmaceutical agents for the modulation of CIS activity.

Methods to assay for protein-protein interactions, such as that of a CIS gene product/binding partner complex, and to isolate proteins interacting with CIS are known to those skilled in the art. Use of the methods discussed below enable one of ordinary skill in the art to accomplish these aims without undue experimentation.

The yeast two-hybrid system provides methods for detecting the interaction between a first test protein and a second test protein, in vivo, using reconstitution of the activity of a transcriptional activator. The method is disclosed in U.S. Pat. No. 5,283,173; reagents are available from Clontech and Stratagene. Briefly, CIS cDNA is fused to a Ga14 transcription factor DNA binding domain and expressed in yeast cells. cDNA library members obtained from cells of interest are fused to a transactivation domain of Ga14. cDNA clones which express proteins which can interact with CIS will lead to reconstitution of Ga14 activity and transactivation of expression of a reporter gene such as Gall-lacZ. Optionally, the host cells can be co-transfected with a protein tyrosine kinase to induce tyrosine phosphorylation of members of the cDNA library. Such phosphorylation is necessary for optimum interaction with the SH2 domain of CIS.

An alternative method is screening of λgt11, λZAP (Stratagene) or equivalent cDNA expression libraries with recombinant CIS. Recombinant CIS protein or fragments thereof are fused to small peptide tags such as FLAG, HSV or GST. The peptide tags can possess convenient phosphorylation sites for a kinase such as heart muscle creatine kinase or they can be biotinylated. Recombinant CIS can be phosphorylated with ³²[p] or used unlabeled and detected with streptavidin or antibodies against the tags. λgt11cDNA expression libraries are made from cells of interest and are incubated with the recombinant CIS, washed and cDNA clones isolated which interact with CIS. See, e.g., T. Maniatis et al, supra.

Another method is the screening of a mammalian expression library in which the cDNAs are cloned into a vector between a mammalian promoter and polyadenylation site and transiently transfected in COS or 293 cells followed by detection of the binding protein 48 hours later by incubation of fixed and washed cells with a labelled CIS, prefereably iodinated, and detection of bound CIS by autoradiography (See Sims et al., Science 241, 585-589 (1988) and McMahan et al., EMBO J. 10, 2821-2832 (1991)). In this manner, pools of cDNAs containing the cDNA encoding the binding protein of interest can be selected and the cDNA of interest can be isolated by further subdivision of each pool followed by cycles of transient transfection, binding and autoradiography. Alternatively, the cDNA of interest can be isolated by transfecting the entire cDNA library into mammalian cells and panning the cells on a dish containing CIS bound to the plate. Cells which attach after washing are lysed and the plasmid. DNA isolated, amplified in bacteria, and the cycle of transfection and panning repeated until a single cDNA clone is obtained (See Seed et al, Proc. Natl. Acad. Sci. USA 84, 3365 (1987) and Aruffo et al., EMBO J. 6, 3313 (1987)). If the binding protein is secreted, its cDNA can be obtained by a similar pooling strategy once a binding or neutralizing assay has been established for assaying supernatants from transiently transfected cells. General methods for screening supernatants are disclosed in Wong et al., Science 228, 810-815 (1985).

Another alternative method is isolation of proteins interacting with CIS directly from cells. Fusion proteins of CIS with GST or small peptide tags are made and immobilized on beads. Biosynthetically labeled or unlabeled protein extracts from the cells of interest are prepared, incubated with the beads and washed with buffer. Proteins interacting with CIS are eluted specifically from the beads and analyzed by SDS-PAGE. Binding partner primary amino acid sequence data are obtained by microsequencing. Optionally, the cells can be treated with agents that induce a functional response such as tyrosine phosphorylation of cellular proteins. An example of such an agent would be a growth factor or cytokine such as erythropoietin or interleukin-3.

Another alternative method is immunoaffinity purification. Recombinant CIS is incubated with labeled or unlabeled cell extracts and immunoprecipitated with anti-CIS antibodies. The immunoprecipitate is recovered with protein A-Sepharose and analyzed by SDS-PAGE. Unlabelled proteins are labeled by biotinylation and detected on SDS gels with streptavidin. Binding partner proteins are analyzed by microsequencing. Further, standard biochemical purification steps known to those skilled in the art may be used prior to microsequencing.

Yet another alternative method is screening of peptide libraries for binding partners. Recombinant tagged or labeled CIS is used to select peptides from a peptide or phosphopeptide library which interact with CIS. Sequencing of the peptides leads to identification of consensus peptide sequences which might be found in interacting proteins.

CIS binding partners identified by any of these methods or other methods which would be known to those of ordinary skill in the art as well as those putative binding partners discussed above can be used in the assay method of the invention. Assaying for the presence of CIS/binding partner complex are accomplished by, for example, the yeast two-hybrid system, ELISA or immunoassays using antibodies specific for the complex. In the presence of test substances which interrupt or inhibit formation of CIS/binding partner interaction, a decreased amount of complex will be determined relative to a control lacking the test substance.

Assays for free CIS or binding partner are accomplished by, for example, ELISA or immunoassay using specific antibodies or by incubation of radiolabeled CIS with cells or cell membranes followed by centrifugation or filter separation steps. In the presence of test substances which interrupt or inhibit formation of CIS/binding partner interaction, an increased amount of free CIS or free binding partner will be determined relative to a control lacking the test substance.

Another aspect of the invention is pharmaceutical compositions comprising an effective amount of a CIS modulator of the invention and a pharmaceutically acceptable carrier. Pharmaceutical compositions of modulators of this invention for parenteral administration, i.e., subcutaneously, intramuscularly or intravenously or oral administration can be prepared.

The compositions for parenteral administration will commonly comprise a solution of the modulators of the invention or a cocktail thereof dissolved in an acceptable carrier, preferably an aqueous carrier. A variety of aqueous carriers may be employed, e.g., water, buffered water, 0.4% saline, 0.3% glycine and the like. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the modulator of the invention in such pharmaceutical formulation can vary widely, i.e., from less than about 0.5%, usually at or at least about 1% to as much as 15 or 20% by weight and will be selected primarily based on fluid volumes, viscosities, etc. according to the particular mode of administration selected.

Thus, a pharmaceutical composition of the modulator of the invention for intramuscular injection could be prepared to contain 1 mL sterile buffered water, and 50 mg of a protein of the invention. Similarly, a pharmaceutical composition of the modulator of the invention for intravenous infusion could be made up to contain 250 mL of sterile Ringer's solution, and 150 mg of a modulator of the invention. Actual methods for preparing parenterally administrable compositions are well known or will be apparent to those skilled in the art and are described in more detail in, for example, Remington's Pharmaceutical Science, 15th ed., Mack Publishing Company, Easton, Pa.

The physician will determine the dosage of the present therapeutic agents which will be most suitable and it will vary with the form of administration and the particular compound chosen, and furthermore, it will vary with the particular patient under treatment. Generally, the physician will wish to initiate treatment with small dosages substantially less than the optimum dose of the compound and increase the dosage by small increments until the optimum effect under the circumstances is reached. It will generally be found that when the composition is administered orally, larger quantities of the active agent will be required to produce the same effect as a smaller quantity given parenterally. The therapeutic dosage will generally be from 1 to 10 milligrams per day and higher although it may be administered in several different dosage units.

Depending on the patient condition, the pharmaceutical composition of the invention can be administered for prophylactic and/or therapeutic treatments. In therapeutic application, compositions are administered to a patient already suffering from a disease in an amount sufficient to cure or at least partially arrest the disease and its complications. In prophylactic applications, compositions containing the present compounds or a cocktail thereof are administered to a patient not already in a disease state to enhance the patient's resistance to the disease.

Single or multiple administrations of the pharmaceutical compositions can be carried out with dose levels and pattern being selected by the treating physician. In any event, the pharmaceutical composition of the invention should provide a quantity of the modulators of the invention sufficient to effectively treat the patient.

Additionally, some diseases result from inherited defective genes. These genes can be detected by comparing the sequence of the defective gene with that of a normal one. Individuals carrying mutations in the CIS gene may be detected at the DNA level by a variety of techniques. Nucleic acids used for diagnosis (genomic DNA, mRNA, etc.) may be obtained from a patient's cells, such as from blood, urine, saliva or tissue biopsy, e.g., chorionic villi sampling or removal of amniotic fluid cells and autopsy material. The genomic DNA may be used directly for detection or may be amplified enzymatically by using PCR, ligase chain reaction (LCR), strand displacement amplification (SDA), etc. prior to analysis. See, e.g., Saiki et al., Nature, 324, 163-166 (1986), Bej, et al., Crit. Rev. Biochem. Molec. Biol., 26, 301-334 (1991), Birkenmeyer et al., J. Virol. Meth., 35, 117-126 (1991), Van Brunt, J., Bio/Technology, 8, 291-294 (1990)). RNA or cDNA may also be used for the same purpose. As an example, PCR primers complementary to the nucleic acid of the instant invention can be used to identify and analyze CIS mutations. For example, deletions and insertions can be detected by a change in size of the amplified product in comparison to the normal CIS genotype. Point mutations can be identified by hybridizing amplified DNA to rabiolabeled CIS RNA of the invention or alternatively, radiolabelled CIS antisense DNA sequences of the invention. Perfectly matched sequences can be distinguished from mismatched duplexes by RNase A digestion or by differences in melting temperatures (Tm). Such a diagnostic would be particularly useful for prenatal and even neonatal testing.

In addition, point mutations and other sequence differences between the reference gene and “mutant” genes can be identified by yet other well-known techniques, e.g., direct DNA sequencing, single-strand conformational polymorphism. See Orita et al., Genomics, 5, 874-879 (1989). For example, a sequencing primer is used with double-stranded PCR product or a single-stranded template molecule generated by a modified PCR. The sequence determination is performed by conventional procedures with radiolabeled nucleotides or by automatic sequencing procedures with fluorescent-tags. Cloned DNA segments may also be used as probes to detect specific DNA segments. The sensitivity of this method is greatly enhanced when combined with PCR. The presence of nucleotide repeats may correlate to a causative change in CIS activity or serve as marker for various polymorphisms.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures. See, e.g., Myers et al., Science, 230, 1242 (1985). In addition, sequence alterations, in particular small deletions, may be detected as changes in the migration pattern of DNA heteroduplexes in non-denaturing gel electrophoresis such as heteroduplex electrophoresis. See, e.g., Nagamine et al., Am. J. Hum. Genet., 45, 337-339 (1989). Sequence changes at specific locations may also be revealed by nuclease protection assays, such as RNase and S1 protection or the chemical, cleavage method as disclosed by Cotton et al. in Proc. Natl. Acad. Sci. USA, 85, 4397-4401 (1985).

Thus, the detection of a specific DNA sequence may be achieved by methods such as hybridization (e.g., heteroduplex electroporation, see, White et al., Genomics, 12, 301-306 (1992), RNAse protection (e.g., Myers et al., Science, 230, 1242 (1985)) chemical cleavage (e.g., Cotton et al., Proc. Natl. Acad. Sci. USA, 85, 4397-4401 (1985)), direct DNA sequencing, or the use of restriction enzymes (e.g., restriction fragment length polymorphisms (RFLP) in which variations in the number and size of restriction fragments can indicate insertions, deletions, presence of nucleotide repeats and any other mutation which creates or destroys an endonuclease restriction sequence). Southen blotting of genomic DNA may also be used to identify large (i.e., greater than 100 base pair) deletions and insertions.

In addition to conventional gel electrophoresis and DNA sequencing, mutations such as microdeletions, aneuploidies, translocations, inversions, can also be detected by in situ analysis. See, e.g., Keller et al., DNA Probes, 2nd Ed., Stockton Press, New York, N.Y., USA (1993). That is, DNA or RNA sequences in cells can be analyzed for mutations without isolation and/or immobilization onto a membrane. Fluorescence in situ hybridization (FISH) is presently the most commonly applied method and numerous reviews of FISH have appeared. See, e.g., Trachuck et al., Science, 250, 559-562 (1990), and Trask et al., Trends, Genet., 7, 149-154 (1991). Hence, by using nucleic acids based on the structure of the CIS genes, one can develop diagnostic tests for genetic mutations.

In addition, some diseases are a result of, or are characterized by, changes in gene expression which can be detected by changes in the mRNA. Alternatively, the CIS gene can be used as a reference to identify individuals expressing an increased or decreased level of CIS protein, e.g., by Northern blotting or in situ hybridization.

Defining appropriate hybridization conditions is within the skill of the art. See, e.g., “Current Protocols in Mol. Biol.” Vol. I & II, Wiley Interscience. Ausbel et al. (eds.) (1992). Probing technology is well known in the art and it is appreciated that the size of the probes can vary widely but it is preferred that the probe be at least 15 nucleotides in length. It is also appreciated that such probes can be and are preferably labeled with an analytically detectable reagent to facilitate identification of the probe. Useful reagents include but are not limited to radioisotopes, fluorescent dyes or enzymes capable of catalyzing the formation of a detectable product. As a general rule, the more stringent the hybridization conditions the more closely related genes will be that are recovered.

The putative role of CIS in signal transduction of the DNA synthesis pathway establishes yet another aspect of the invention which is gene therapy. “Gene therapy” means gene supplementation where an additional reference copy of a gene of interest is inserted into a patient's cells. As a result, the protein encoded by the reference gene corrects the defect and permits the cells to function normally, thus alleviating disease symptoms. The reference copy would be a wild-type form of the CIS gene or a gene encoding a protein or peptide which modulates the activity of the endogenous CIS.

Gene therapy of the present invention can occur in vivo or ex vivo. Ex vivo gene therapy requires the isolation and purification of patient cells, the introduction of a therapeutic gene and introduction of the genetically altered cells back into the patient. A replication-deficient virus such as a modified retrovirus can be used to introduce the therapeutic CIS gene into such cells. For example, mouse Moloney leukemia virus (MMLV) is a well-known vector in clinical gene therapy trials. See, e.g., Boris-Lauerie et al., Curr. Opin. Cenet. Dev., 3, 102-109 (1993).

In contrast, in vivo gene therapy does not require isolation and purification of a patient's cells. The therapeutic gene is typically “packaged” for administration to a patient such as in liposomes or in a replication-deficient virus such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. Another approach is administration of “naked DNA” in which the therapeutic gene is directly injected into the bloodstream or muscle tissue. Another approach is administration of “naked DNA” in which the therapeutic gene is introduced into the target tissue by microparticle bombardment using gold particles coated with the DNA.

Cell types useful for gene therapy of the present invention include lymphocytes, hepatocytes, myoblasts, fibroblasts, any cell of the eye such as retinal cells, epithelial and endothelial cells. Preferably the cells are T lymphocytes drawn from the patient to be treated, hepatocytes, any cell of the eye or respiratory or pulmonary epithelial cells. Transfection of pulmonary epithelial cells can occur via inhalation of a neubulized preparation of DNA vectors in liposomes, DNA-protein complexes or replication-deficient adenoviruses. See, e.g., U.S. Pat. No. 5,240,846.

Another aspect of the invention is transgenic, non-human mammals capable of expressing the polynucleotides of the invention in any cell. Transgenic, non-human animals may be obtained by transfecting appropriate fertilized eggs or embryos of a host with the polynucleotides of the invention or with mutant forms found in human diseases. See, e.g., U.S. Pat. Nos. 4,736,866; 5,175,385; 5,175,384 and 5,175,386. The resultant transgenic animal may be used as a model for the study of CIS gene function. Particularly useful transgenic animals are those which display a detectable phenotype associated with the expression of the CIS protein. Drug development candidates may then be screened for their ability to reverse or exacerbate the relevant phenotype.

The present invention will now be described with reference to the following specific, non-limiting examples.

### EXAMPLE 1

### Cis Full-Length cDNA Cloning And Sequence Analysis

A search of a random cDNA sequence database consisting of short partial sequences known as expressed sequence tags (ESTs) with SH2 domain encoding cDNA sequences using the BLASTX algorithm disclosed an EST which was homologous to the SH2 domain of the regulatory subunit of PI-3 kinase. This EST was originally isolated from a human endometrial tumor cDNA library. Further searching revealed that the EST was homologous to the 3′ end of a murine CIS cDNA sequence reported by A. Yoshimura et al., supra, (Genbank Accession No. D31943) (SEQ ID NO:3)

A circular 5′-rapid amplification of cDNA ends (cRACE) protocol as described by Maruyama et al. in Nucl. Acids Res. 23, 3796-3797 (1995) was used to isolate the 5′ cDNA end of the putative human CIS. One hundred ng of human skeletal muscle polyA RNA (Clontech #6541-1) was reverse transcribed with MoMLV reverse transcriptase using a 5′-phosphorylated gene-specific primer GGCCACATAGTGCTGCACAA (SEQ ID NO:5). The single-stranded cDNA product was circularized by treatment with T4 RNA ligase. Two adjacent gene-specific primers GGAAGCTGGAGTCGGCATAC (SEQ ID NO:6) and CTCCAACTGCTTGTCCAGGC (SEQ ID NO:7), priming in opposite directions, were used to amplify by PCR a 0.5 kb fragment from the single-stranded circular cDNA template. PCR was conducted at 94° C. for 20 s, 60° C. to 40° C. in 0.5° C. increment/cycle for 30 s, 72° C. for 2 min., for 40 cycles. The 0.5 kb 5′ cRACE fragment was subcloned into pBluescript II and sequenced.

Sequence analysis revealed the fragment to be the 5′-end of the 3′ clone of the putative human CIS, containing its remaining coding sequence, including the N-terminus. The encoded protein contains a central SH2 domain flanked by domains of unknown function.

A cDNA encoding an intact coding sequence was assembled. A 1.6 kb fragment was amplified from the 3′ clone by PCR using the primer ACAGCACGCACCCCAGCTAC (SEQ ID NO:8) and the T7 primer. Similarly, a 0.5 kb fragment was amplified from the 5′ cRACE product, isolated as described above, using the primers GGAAGCTGGAGTCGGCATAC (SEQ ID NO:6) and CTCCAACTGCTTGTCCAGGC (SEQ ID NO:7). These products were recombined by PCR in a second reaction containing each of the above PCR products and the primers GGAATTCCATGGTCCTCTGCGTTCAGGG (SEQ ID NO:9) and CCGTCGACGGTCAGAGCTGGAAGGGGTACT (SEQ ID NO:10). The PCR conditions for both sets of reactions were 94° C. for 15 s, 55° C. for 20 s, 72° C. for 1 min., for 25 cycles. The 0.8 kb secondary PCR product was treated with EcoRI and SalI and subcloned into pBluescript II (Stratagene) and pGEX4T-3 (Pharmacia). High levels of CIS protein expression in bacteria were observed with the pGEX4T-3 construct.

Independent confirmation of the existence of a mRNA corresponding to the full-length cDNA produced was carried out by RT-PCR. cDNA was prepared from 100 ng of human skeletal muscle polyA RNA (Clontech #6541-1) using random hexamer primers and MoMLV reverse transciptase. One twentieth of the cDNA was used as template in a PCR reaction containing the primers ATGGTCCTCTGCGTTCAGGG (SEQ ID NO:11) and TCAGAGCTGGAAGGGGTACT (SEQ ID NO:12) and the expected 786 bp product was observed. The PCR conditions were 94° C. for 15 s, 70° C. to 50° C. in 0.5° C. increment/cycle for 20 s, 72° C. for 2 min., for 40 cycles. Control reactions containing no template or containing the 0.8 kb recombined cDNA produced above gave either no PCR product or a 786 bp product, as expected.

Sequence analysis of the entire human CIS cDNA revealed a 774 nucleotide open reading frame (SEQ ID NO:1) encoding a 258 amino acid protein (SEQ ID NO:2) with a predicted molecular mass of 28.4 kDa, starting with an ATG at position 72 and terminating with a TGA at position 846 of SEQ ID NO:1. A proline-rich region is present at the C-terminus. The SH2 domain of human CIS is encoded by nucleotides 315 to 618 which correspond to residues 82 (Trp) to 183 (Val) in SEQ ID NO:2. GenBank searches using the BLASTX and BLASTP algorithms with the full-length DNA sequence or with the deduced amino acid sequence indicated that the human CIS SH2 domain was most homologous with the SH2 domain of the regulatory subunit of PI-3 kinase.

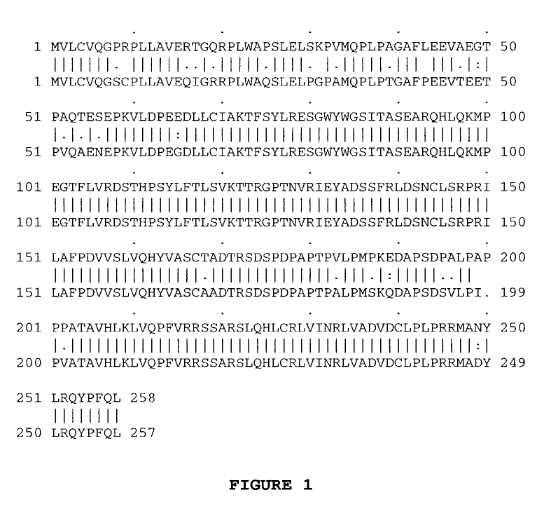

Alignment of the deduced amino acid sequence of human CIS (SEQ ID NO:2) with the murine CIS amino acid sequence (SEQ ID NO:4) was accomplished using the GAP algorithm. The overall amino acid identity was 90% with a one amino acid gap and is shown in FIG. 1 (top, human CIS; bottom, murine CIS).

### EXAMPLE 2

### Tissue Distribution of CIS

Northern blots of tissue mRNA were conducted to determine the tissue distribution of CIS gene transcripts. The 1.8 kb insert of the 3° CIS clone was PCR amplified using T3 and T7 primers. Twenty five ng of the isolated PCR product was radiolabelled with [32P]-dATP using a randomly primed labelling kit from Stratagene.

Membranes containing mRNA from multiple human tissues (Clontech #7760-1 and #7759-1) were hybridized with the probes and washed under high stringency conditions. Hybridized mRNA was visualized by exposing the membranes for six hours to a storage phosphor screen (Molecular Dynamics). The results indicated that the 2.2 kb CIS transcript is largely ubiquitous and is expressed at variable levels in heart, placenta, lung, liver, muscle, kidney, pancreas, spleen, thymus, prostate, testis, ovary, intestine, colon and peripheral blood lymphocytes. Highest expression was observed in liver and ovary. The mRNA appears absent from brain.

The present invention may be embodied in other specific forms without departing from the spirit or essential attributes thereof, and, accordingly, reference should be made to the appended claims, rather than to the foregoing specification, as indicating the scope of the invention.

1. An isolated polynucleotide selected from the group consisting of:
(a) a polynucleotide encoding human CIS having the nucleotide sequence as set forth in SEQ ID NO: 1 from nucleotide 72 to 846;
(b) a polynucleotide capable of hybridizing to the complement of a polynucleotide according to (a) under moderately stringent hybridization conditions and which encodes a functional human CIS; and
(c) a degenerate polynucleotide according to (a) or (b).

2. An isolated polynucleotide having the nucleotide sequence as set forth in SEQ ID NO:1.

3. A functional polypeptide encoded by the polynucleotide of claim 1.

4. The functional polypeptide of claim 3 which is human CIS having the amino acid sequence set forth in SEQ ID NO:2.

5. The polynucleotide of claim 1 which is DNA.

6. The polynucleotide of claim 5 which is genomic DNA.

7. The polynucleotide of claim 1 which is RNA.

8. A vector comprising the DNA of claim 5.

9. A recombinant host cell comprising the vector of claim 8.

10. A method for preparing essentially pure human CIS protein comprising culturing the recombinant host cell of claim 9 under conditions promoting expression of the protein and recovering the expressed protein.

11. Human CIS produced by the process of claim 10.

12. An antisense oligonucleotide comprising a sequence which is capable of binding to the polynucleotide of claim 1.

13. A modulator of the polypeptide of claim 3.

14. The modulator of claim 13 which is a peptide.

15. The modulator of claim 13 which is a small organic molecule.

16. The small organic molecule of claim 15 which is a peptidomimetic.

17. A method for assaying a medium for the presence of a substance that modulates CIS activity by affecting the binding of CIS to cellular binding partners comprising the steps of:
(a) providing a CIS protein having the amino acid sequence of CIS (SEQ ID NO:2) or a functional derivative thereof and a cellular binding partner or synthetic analog thereof;
(b) incubating with a test substance which is suspected of modulating CIS activity under conditions which permit the formation of a CIS protein/cellular binding partner complex;
(c) assaying for the presence of the complex, free CIS protein or free cellular binding partner; and
(d) comparing to a control to determine the effect of the substance.

18. CIS protein modulating compounds identified by the method of claim 17.

19. A method for the treatment of a patient having need to modulate CIS activity comprising administering to the patient a therapeutically effective amount of the modulating compound of claims 18.

20. A pharmaceutical composition comprising the modulating compound of claim 18 and a pharmaceutically acceptable carrier.

21. A method for assaying for the presence of a substance that modulates CIS activity by direct binding to CIS protein comprising the steps of:
(a) providing a labelled CIS protein having the amino acid sequence of CIS (SEQ ID NO:2) or a functional derivative thereof
(b) providing solid support-associated modulator candidates;
(c) incubating a mixture of the labelled CIS protein with the support-associated modulator candidates under conditions which can permit the formation of a CIS protein/modulator candidate complex;
(d) separating the solid support from free soluble labelled CIS protein;
(e) assaying for the presence of solid support-associated labelled protein;
(f) isolating the solid support complexed with labelled CIS protein; and
(g) identifying the modulator candidate.

22. CIS protein modulating compounds identified by the method of claim 21.

23. A method for the treatment of a patient having need to modulate CIS activity comprising administering to the patient a therapeutically effective amount of the modulating compound of claim 21.

24. A pharmaceutical composition comprising the modulating compound of claim 21 and a pharmaceutically acceptable carrier.

25. A method of diagnosing conditions associated with CIS protein deficiency which comprises:
(a) isolating a polynucleotide sample from an individual;
(b) assaying the polynucleotide sample and a polynucleotide encoding CIS having the nucleotide sequence as set forth in SEQ ID NO:1 from nucleotide 72 to 846; and
(c) comparing differences between the polynucleotide sample and the CIS polynucleotide, wherein any differences indicate mutations in the CIS gene.

26. A method of treating conditions which are related to insufficient CIS protein function which comprises:
(a) isolating cells from a patient deficient in CIS protein function;
(b) altering the cells by transfecting the polynucleotide of claim 1 into the cells wherein a CIS protein is expressed; and
(c) introducing the cells back to the patient to alleviate the condition.

27. A method of treating conditions which are related to insufficient CIS protein function which comprises administering the polynucleotide of claim 1 to a patient deficient in CIS protein function wherein a CIS protein is expressed and alleviates the condition.

28. A transgenic non-human animal capable of expressing in any cell thereof the DNA of claim 5.


SEQUENCE LISTING (12 sequences)

SEQ ID NO: 1 - DNA (cDNA), 1374 bp; single-stranded, linear
  hypothetical: NO
  anti-sense: NO
  fragment type: <Unknown>
tcctgcactgctgatacccgaagcgacagcccgatcctgctcccactccggagtcgccgctgcgcggagacatggtcctctgcgttcagggacctcgtcctttgctggctgtggagcggactgggcagcggcccctgtgggccccgtccctggaactgtccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggctggtattggggttccattacggccagcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagtcttgtgcagcactatgtggcctcctgcactgctgatacccgaagcgacagccccgatcctgctcccaccccggtcctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccaactacctccgacagtaccccttccagctctgactgtacggggcaatctgccaccctcacccagtcgcaccctggaggggacatcagccccagctggacttgggcccccactgtccctcctccaggcatcctggtgcctgcatacctctggcagctggcccaggaagagccagcaagagcaaggcatgggagaggggaggtgtcacacaacttggaggtaaatgcccccaggccgcatgtggcttcattatactgagccatgtgtcagaggatggggagacaggcaggaccttgtctcacctgtgggctgggcccagacctccactcgattgcctgccctggtcacctgaactgtatgggcactctcagccctggtttttcaatccccagggtcgggtaggacccctactgcgcagccagtctctttttctgggaggatgacatgcagcggaactgagatcgacagtgtactagtgacctcttgttgaggggtaagccaggataggggacttgcacaatctatacactatttatttatttattctccgtgggggttgca

SEQ ID NO: 2 - protein, 258 aa
  hypothetical: NO
  anti-sense: NO
  fragment type: N-terminal
MVLCVQGPRPLLAVERTGQRPLWAPSLELSKPVMQPLPAGAFLEEVAEGTPAQTESEPKVLDPEEDLLCIAKTFSYLRESGWYWGSITASEARQHLQKMPEGTFLVRDSTHPSYLFTLSVKTTRGPTNVRIEYADSSFRLDSNCLSRPRILAFPDVVSLVQHYVASCTADTRSDSPDPAPTPVLPMPKEDAPSDPALPAPPPATAVHLKLVQPFVRRSSARSLQHLCRLVINRLVADVDCLPLPRRMANYLRQYPFQL

SEQ ID NO: 3 - DNA (cDNA), 2000 bp; single-stranded, linear
  hypothetical: NO
  anti-sense: NO
  fragment type: <Unknown>
gtccccccttgtccttccaagctgttcgcaccacagcctttcagtccctgctcgccgcccgtgtgccccgggaccctgaccttcgcacccctggacccattggctcctttctccttccatcccgccgaactccgactctcgagccgccgttgtctctgggacatggtcctctgcgtacagggatcttgtcctttgctggctgtggagcaaattgggcggcggcctctgtgggcccagtccctggagctgcccgggccagccatgcagcccttacccactggggcattcccagaggaagtgacagaggagacccctgtccaggcagagaatgaaccgaaggtgctagaccctgagggggatctgctgtgcatagccaagacgttctcctaccttcgggaatctgggtggtactggggttctattacagccagcgaggcccggcagcacctacagaagatgccggagggtacattcctagttcgagacagcacccaccccagctacctgttcacactgtcagtcaaaaccacccgtggccccaccaacgtgcggatcgagtacgccgattctagcttccggctggactctaactgcttgtcaagacctcgaatcctggccttcccagatgtggtcagccttgtgcagcactatgtggcctcctgtgcagctgacacccggagcgacagcccggatcctgctcccaccccagccctgcctatgtctaagcaagatgcacctagtgactcggtgctgcctatccccgtggctactgcagtgcacctgaaactggtgcagccctttgtgcgcaggagcagtgcccgcagcttacaacatctgtgtcggctagtcatcaaccgtctggtggccgacgtggactgcttacccctgccccggcgtatggccgactacctccgacagtaccccttccaactctgactgagccaggcaccctgctctgcctcacacagtcacatcctggagggaacacagtccccagctggacttggggttctgctgtcctttcttcagtcatcctggtgcctgcatgcatgtgacagctggaccagagaatgccagcaagaacaaggcaggtggaggagggattgtcacacaactctgaggtcaacgcctctaggtacaatatggctctttgtggtgagccatgtatcagagcgagacaggcaggacctcgtctctccacagaggctggacctaggtctccactcacttgcctgcccttgccacctgaactgtgtctattctcccagccctggtttctcagtctgctgagtagggcaggccccctacccatgtatagaatagcgagcctgtttctgggagaatatcagccagaggttgatcatgccaaggccccttatggggacgcagactgggctaggggactacacagttatacagtatttatttatttattctccttgcaggggttgggggtggaatgatggcgtgagccatcccacttctctgccctgtgctctgggtggtccagagacccccaggtctggttcttccctgtggagacccccatcccaaaacattgttgggcccaaagtagtctcgaatgtcctgggcccatccacctgcgtatggatgtgcccacttttttctcccaagcctcttttgggaggctgggtggccagacagacaggagccagaaacacaagggctcccactcttctcctcacagggcagcaccatggcttcatagagctggcttctctatgttgtgccccacctcacccccctgccgaggggcgtgtgctgggtcgggaagtggatgcttatccaagggccgcagatgtagctcccttgtgtccgtttcctgcctaggaagttgcctgcacgtgagagagggagaaatacatacacacctaacaagactttagaaaacaagtgttagaacacaagaaccagtttgggagtttttcttccactgatttttttctgtaatgataataaaattatgccttccacttatg

SEQ ID NO: 4 - protein, 257 aa
  hypothetical: NO
  anti-sense: NO
  fragment type: N-terminal
MVLCVQGSCPLLAVEQIGRRPLWAQSLELPGPAMQPLPTGAFPEEVTEETPVQAENEPKVLDPEGDLLCIAKTFSYLRESGWYWGSITASEARQHLQKMPEGTFLVRDSTHPSYLFTLSVKTTRGPTNVRIEYADSSFRLDSNCLSRPRILAFPDVVSLVQHYVASCAADTRSDSPDPAPTPALPMSKQDAPSDSVLPIPVATAVHLKLVQPFVRRSSARSLQHLCRLVINRLVADVDCLPLPRRMADYLRQYPFQL

SEQ ID NO: 5 - DNA, 20 bp; single-stranded, linear
  hypothetical: NO
  anti-sense: NO
  fragment type: <Unknown>
ggccacatagtgctgcacaa

SEQ ID NO: 6 - DNA, 20 bp; single-stranded, linear
  hypothetical: NO
  anti-sense: NO
  fragment type: <Unknown>
ggaagctggagtcggcatac

SEQ ID NO: 7 - DNA, 20 bp; single-stranded, linear
  hypothetical: NO
  anti-sense: NO
  fragment type: <Unknown>
ctccaactgcttgtccaggc

SEQ ID NO: 8 - DNA, 20 bp; single-stranded, linear
  hypothetical: NO
  anti-sense: NO
  fragment type: <Unknown>
acagcacgcaccccagctac

SEQ ID NO: 9 - DNA, 28 bp; single-stranded, linear
  hypothetical: NO
  anti-sense: NO
  fragment type: <Unknown>
ggaattccatggtcctctgcgttcaggg

SEQ ID NO: 10 - DNA, 30 bp; single-stranded, linear
  hypothetical: NO
  anti-sense: NO
  fragment type: <Unknown>
ccgtcgacggtcagagctggaaggggtact

SEQ ID NO: 11 - DNA, 20 bp; single-stranded, linear
  hypothetical: NO
  anti-sense: NO
  fragment type: <Unknown>
atggtcctctgcgttcaggg

SEQ ID NO: 12 - DNA, 20 bp; single-stranded, linear
  hypothetical: NO
  anti-sense: NO
  fragment type: <Unknown>
tcagagctggaaggggtact